(19) United States
(12) Patent Application Publication
(10) Pub. No.: US 2014/0352692 A1
(43) Pub. Date: Dec. 4, 2014

(54) Dose Counting Mechanism for an Inhalation Device and Inhalation Device

(71) Applicant: Sanofi SA, Vernier (CH)

(72) Inventor: Stefan Mayer, Freiburg im Breisgau (DE)

(21) Appl. No.: 14/372,010

(22) PCT Filed: Jan. 22, 2013
(86) PCT No.: PCT/EP2013/051145

§ 371 Date: Jul. 12, 2014

(30) Foreign Application Priority Data
Jan. 23, 2012 (EP) 12152163.7

(51) Int. Cl. A61M 15/00

(52) U.S. Cl. CPC A61M 15/0076; A61M 15/0091; A61M 15/008
USPC 128/203.12

(57) ABSTRACT

The present disclosure refers to a dose counting mechanism for an inhalation device comprising a first state and a second state wherein the mechanism is alterable from the first state to the second state by a user's suction airstream. Further, the present disclosure concerns an inhalation device comprising said dose counting mechanism.

### CROSS REFERENCE TO RELATED APPLICATIONS

The present application is a U.S. National Phase Application pursuant to 35 U.S.C. §371 of International Application No. PCT/EP2013/051145 filed Jan. 22, 2013, which claims priority to European Patent Application No. 12152163.7 filed Jan. 23, 2012. The entire disclosure contents of these applications are herewith incorporated by reference into the present application.

### FIELD OF INVENTION

This disclosure relates to a dose counting mechanism for an inhalation device and to an inhalation device comprising such a dose counting mechanism.

### BACKGROUND

An inhalation device is usually activated by the user's suction airstream and is intended for the inhalation of a substance, in particular a powdery substance. An inhalation device is described in document WO 2009/065707 A1, for example.

Inhalation devices comprising a dose counting mechanism are known in the prior art. In particular, WO 2009/029028 A1 shows an inhaler comprising an inhalation-triggered release mechanism and a dose counter. Further, WO 2007/141520 A1 shows a breath-activated metered dose inhaler comprising a dose counter for automatically counting the remaining doses left in a container. Moreover, EP 1 386 630 A1 discloses a powder inhaler comprising a dosing sub-assembly and a dose counting sub-assembly.

### SUMMARY

It is an object of the present disclosure to provide a dose counting mechanism for an improved inhalation device, e.g. a user-friendly dose counting mechanism. Furthermore, it is an object of the present disclosure to provide an improved inhalation device.

This object may, inter alia, be achieved by the subjects of the independent claims. Advantageous embodiments and refinements are the subject-matter of the dependent claims. However, further advantageous concepts may be disclosed herein besides the ones which are claimed.

One aspect relates to a dose counting mechanism for an inhalation device comprising a first state and a second state wherein the mechanism is configured to change from the first state to the second state by a user's suction airstream.

For operation of the dose counting mechanism, an interaction member may be used, the interaction member being comprised by the mechanism. The interaction member is enabled to operate a counting function of the dose counting mechanism. This may happen in particular by interaction of the interaction member with other parts of the mechanism. In particular, the interaction member may interact with other parts of the mechanism in order to increment or decrement the counted number.

The state of the dose counting mechanism may correspond to a relative location of parts of the dose counting mechanism to each other. In particular, two parts of the dose counting mechanism may be in engagement to each other in one of the first and the second state of the dose counting mechanism. Further, said two parts may be disengaged from each other in the other of the first and the second state of the dose counting mechanism. The two parts may be an interaction member and a counting member.

In particular, the second state may be an activated state of the counting mechanism. Further, the first state may be a deactivated state of the counting mechanism.

The second state of the dose counting mechanism may be a state wherein the dose counting mechanism is enabled to increment or decrement a counted number. Further, the first state may be a state wherein the dose counting mechanism is prevented from incrementing a counted number without prior activation.

The present disclosure further relates to an inhalation device comprising a dose counting mechanism and an actuating element wherein the actuating element is configured to be moved during a dose delivery operation of the inhalation device,

wherein the counting mechanism has a first state and a second state and wherein the counting mechanism is configured to change from the first state to the second state by a user's suction airstream, wherein the dose counting mechanism comprises an interaction member and a counting member, wherein the counting member counts a counted number, wherein the interaction member is enabled to interact with the counting member for incrementing or decrementing the counted number, and

wherein the interaction member is an integral part of the actuating element of the inhalation device or may be fixed to the actuating element.

The actuating element which is moved during a dose delivery operation of the inhalation device may be part of a dose delivery mechanism. The actuating element may be required in the inhalation device for purposes other than the counting mechanism. The actuating element may be configured to be moved by a user's suction airstream in a dose delivery operation. A movement of the actuating element may be required in the dose delivery operation.

As the counting mechanism may be configured to cooperate with the actuating element, the number of components required for the counting mechanism may be reduced. In particular, the number of components required for the counting mechanism may be reduced as the interaction member is fixed to or even integrally formed by the actuating element. In particular, a movement of the actuating element may alter the state of the counting mechanism. A movement of the actuating element may be transferred by the interaction member to further elements of the counting mechanism. For example, the interaction member may be configured to transfer a movement of the actuating element into a movement of the counting member.

The dose counting mechanism may be configured to increment or decrement a number. In particular, the counted number may correspond to the number of doses left in the device. In this case, after each dose delivery, the counted number may be decremented by one.

The dose counting mechanism may be an assembly, in particular an assembly of several parts.

Preferably, the dose counting mechanism is configured such that an increment or decrement of the counted number is only enabled if a dose delivery has actually been carried out by the inhalation device. In particular, the inhalation device may be configured such that a user's suction airstream causes a delivery of a dose, such that the airstream may be regarded as corresponding to a delivery.

However, after performing a first suction corresponding to the delivery of a dose the user may suck at a mouthpiece of the inhalation device again without having loaded the inhalation device, e.g. by engaging and afterwards disengaging an engaging member to a housing of the device. This second suction may not trigger a second dose delivery as the inhalation is not loaded. Preferably, the dose counting mechanism is configured such that the dose counting mechanism does not change the counted number in this case. Preferably, the user's suction alters the state of the dose counting mechanism to the second state, but does not actually lead to a change of the counted number.

Thereby, it is ensured that a second suction does not trigger a miscounting. In particular, a second suction performed at an unloaded inhalation device does not alter the state of the dose counting mechanism.

In one embodiment, the counting mechanism is enabled to count, i.e. increment or decrement a counted number, only when the counting mechanism is in its second state. In other words, the counting mechanism must have previously been brought into it's activated, second state for an increment. Accordingly, when the counting mechanism is in its first state, the counting mechanism is prevented from incrementing or decrementing a counted number. Thereby, it is ensured that an accidental counting does not occur.

The incrementing or decrementing of the counted number may be caused by engaging an engaging member, e.g. a cap, to the housing of the device.

In one embodiment, the counting mechanism is configured such that a change from its second state to its first state increments or decrements the counted number. In particular, the counting mechanism may be changed to its first state by engaging an engaging member, for example a cap member, to a housing of the inhalation device.

The counted number is only incremented or decremented by engaging the engaging member to the housing if the counting mechanism had been altered to the second state beforehand. Thereby, a miscounting is prevented in the following scenario: A user disengages the cap member from the housing. The user does not perform an inhalation. Accordingly, no dose is delivered. Then the user engages the cap member to the housing. The mere disengagement and engagement of the cap member does not alter the state of the dose counting mechanism. In particular, the dose counting mechanism remains in its first state, in particular a deactivated state. Therefore, engaging the cap member to the housing does not alter the state of the dose counting mechanism and, accordingly, does not lead to a change of the counted number. If a dose had been delivered before, the engagement of the cap member would lead to a change of the counted number. In particular, the dose counting mechanism would be altered from its second state to its first state, thereby changing the counted number.

In other words, the dose counting mechanism is constructed such that the counted number is incremented only if in a first step the engaging member is disengaged from the housing, in a second step the user performs a suction at the mouthpiece and in the third step the engaging member is engaged to the housing. Thereby, the dose counting mechanism prevents a miscounting in the following two scenarios:

The user may skip the second step, i.e. the engaging member is disengaged and afterwards reengaged to the housing without a dose being dispensed. The dose counting mechanim is configured such that in this scenario, an incrementing or a decrementing of the counted number is not caused. In the second scenario, the user performs two suctions in the second step. However, in this case, only one dose is delivered. The dose counting mechanism is configured such that the counted number is incremented or decremented only once in this scenario.

The inhalation device comprises an element that is moved during a dose delivery operation, i.e. the actuating element which is moved axially in a distal direction during the dose delivery operation. In particular, the actuating element is moved by the user's suction airstream. Further, the dose counting mechanism comprises an interaction member which is fixed to the actuating element. Accordingly, the interaction member follows an axial movement of the actuating element. Further, the counted number of the dose counting mechanism may only be altered if the interaction member has been displaced by a movement of the actuating element previously.

The second state of the counting mechanism corresponds to the interaction member being moved by a movement of the actuating element due to a dose delivery. The first state of the counting mechanism accordingly corresponds to the interaction member and the actuating element being in a first position before a dose delivery is performed.

Further, when an engaging member is engaged to the housing and a dose has been delivered previously, during this engagement process, the actuating element and thereby the interaction member are moved. This movement corresponds to an incrementing or decrementing of the counted number.

In one embodiment, the counting mechanism comprises a counting member and an interaction member enabled to interact with the counting member for changing a counted number. The counting member may comprise a toothed wheel. The interaction member may interact with the counting member either directly or via a coupling member.

In this case, the interaction member may be enabled to cause a rotation of the wheel. Preferably, the toothed wheel comprises digits which may correspond to the number of doses left in the device. The dose counting mechanism may be configured such that the interaction member and the toothed wheel interact such that the toothed wheel is configured to display the number of doses left in the device.

The counting mechanism may comprise a plurality of wheels. In particular, the counting mechanism may comprise a first toothed wheel, a second wheel and a third wheel. The first toothed wheel may correspond to the units digit of the number of left doses, the second wheel may correspond to the tens digit and the third wheel may correspond to the hundreds digit. The interaction member may be configured to directly interact with the first toothed wheel. The first toothed wheel may further be configured to directly interact with the second wheel. Moreover, the second wheel may be configured to directly interact with the third wheel.

The second and the third wheel may be toothed as well. However, other couplings between the wheels are also possible.

Further, other embodiments of the wheels are possible as well. In particular, the dose counting mechanism may comprise only one or two wheels comprising digits. Alternatively, the first toothed wheel may be coupled to another display unit, e.g. an electronic or mechanic display which transforms a rotation of the toothed wheel into an update of the display unit.

In one embodiment, the dose counting mechanism may further comprise a coupling member wherein the interaction member is configured to act on the coupling member and the coupling member is enabled to transmit a movement of the interaction member to the counting member. In particular, the coupling member may be Geneva wheel or a toothed wheel.

Further, the coupling member may comprise a toothed wheel, and the interaction member may be configured to slide along a tooth of the toothed wheel when the counting mechanism is altered from the first state to the second state.

In one embodiment, the coupling member and the counting member are formed by a single element. In other words, the coupling member may be provided as an integral part of the counting member.

In one embodiment, the interaction member is configured to slide along a tooth of the toothed wheel when the counting mechanism is changed from the first to the second state, in particular along a top face of a tooth of the toothed wheel. Preferably, by sliding along the side of a tooth, the toothed wheel is not moved. However, the interaction member may be transferred into a position where it is ready to move the toothed wheel.

In one embodiment, the mechanism is configured such that the interaction member acts on a tooth when the counting mechanism is altered from the second to the first state, thereby incrementing or decrementing the counted number. In particular, the interaction member may push the tooth. Thereby, the interaction member may rotate the first toothed wheel by a predetermined angle.

The teeth of the toothed wheel may comprise two interaction faces each. The first interaction face may be a top face of the tooth. The second interaction face may be a side face of the tooth. The dose counting mechanism may be configured such that the interaction member may slide along the top face without moving the toothed wheel. In particular, the top face of the tooth abutting the interaction member may be inclined with respect to a longitudinal axis of the inhalation device and the longitudinal axis of the interaction member when the counting mechanism is in its first state.

Moreover, the side face may be configured such that the interaction member may act on the side face, i.e. transfer a force to the side face. The force may be directed tangentially, in particular perpendicular, to the rotational axis of the first toothed wheel. Thereby, the force may cause a rotation of the toothed wheel. In particular, the side face of the tooth abutting the interaction member may be substantially perpendicular to a longitudinal axis of the interaction member and the longitudinal axis of the inhalation device when the counting mechanism has been brought into its second state.

Further, a first toothed wheel may be enabled to interact with a second and third wheel such that after a complete rotation of the first toothed wheel, the second wheel is rotated by a predetermined angle. Further, after a complete rotation of the second wheel, the third wheel may be rotated by a predetermined angle.

In one embodiment, the interaction member is deflectable. In particular, the interaction member may comprise a deflectable stick. The interaction member may be formed by plastic material. In particular, the interaction member may be formed by injection molding and may be configured to be engaged with an actuating element of an inhalation device or may be an integral part of an actuating element. Preferably, the interaction member comprises a light weight such that it does not significantly increase the force required to move the actuating element. The actuating element may comprise a piston.

In one embodiment, the dose counting mechanism may be configured such that the interaction member is deflected in the first state of the counting mechanism. Further, in one embodiment the interaction member is undeflected in the second state of the counting mechanism. Accordingly, altering the counting mechanism from the first state to the second state may correspond to shifting the interaction member from a first state to a second state, in particular from a deflected state into an undeflected state.

In one embodiment, the dose counting mechanism further comprises a spring for adjusting an amount of underpressure required for activating the inhalation device. The spring may be coupled to the interaction member or to an actuating element of the inhalation device.

Preferably, the inhalation device is activated by the user's suction airstream. As the user's suction airstream also alters the counting mechanism from its first state to its second state, the required force applied by the user's suction airstream to activate the inhalation device may be increased by the dose counting mechanism. This effect may be evened out by the spring. In particular, the spring may apply a force to the actuating element which is directed opposite to the force applied to the actuating element by the interaction member, e.g. the spring may apply a force to the piston towards a mouthpiece of the inhalation device.

According to one aspect, the present disclosure refers to an inhalation device comprising a dose counting mechanism as disclosed above. In particular, said dose counting mechanism may be coupled to an actuating element of the inhalation device. The actuating element may comprise a piston. Particularly, the actuating element may be configured as a breath actuator that is actuatable by a user's suction airstream.

In one embodiment, the inhalation device may further comprise a housing and an engaging member that is engageable to the housing. The counting mechanism may be in its first state when the engaging member is engaged to the housing. By engaging the engaging member to the housing the counting mechanism may be altered from the second state to the first state if the counting mechanism is in its second state before engaging the engaging member to the housing. If the counting mechanism is in its first state before engaging the engaging member to the housing, the counting mechanism remains in its first state when the engaging member is engaged to the housing. As an example, the engaging member may be a cap.

Preferably, when the cap is engaged with the housing, a delivery of a dose is not possible. Accordingly, with the cap engaged there is no need for the counting mechanism to be activated, i.e. altered to its second state, as an increment of the counted dose is only required if a dose is dispensed. In particular, the dose counting mechanism and the inhalation device may be configured such that the correct number of doses left in the device is shown by the counting mechanism when the cap is disengaged from the housing.

In one embodiment, the inhalation device comprises an actuating element being displaceable from a first position to a second position by the user's suction airstream. The first position may be a more distal position than the second position. In particular, the first position may be closer to a mouthpiece of the inhalation device than the second position.

Preferably, the counting mechanism is coupled to the actuating element and alterable form its first state to its second state by a displacement of the actuating element. Vice versa, the counting mechanism may be alterable form its second state to its first state by a displacement of the actuating element. If the actuating element is in its first position, a delivery of a dose may be prevented. If the actuating element is in its second position, a delivery of a dose may be enabled.

Preferably, the interaction member of the counting mechanism is coupled to the actuating element and alterable form its first state to its second state by a displacement of the actuating element. In particular, the interaction member may be coupled to the actuating element such that the interaction member follows an axial movement of the actuating element during a dose delivery operation.

Furthermore, engaging the cap to the housing may move the actuating element from its second position to its first position. Preferably, the inhalation device is configured such that a dose delivery is only performed if the cap is engaged to the housing between a first suction and a successive second suction.

In one embodiment, the counting mechanism may be configured to change from the first state to the second state by displacing the actuating element from the first position to the second position. The actuating element may be displaced by a user's suction airstream.

Preferably, the counted dose is incremented or decremented only when the dose counting mechanism has been altered to its second state and, afterwards, the cap is put back onto the housing. Thereby, an erroneous double counting of two suctions without engaging the cap to the housing in between is prevented. In this case, only the first suction corresponds to a dose delivery. The second suction does not trigger a dose delivery.

In one embodiment, the inhalation device may comprise a balancing member. In particular, both the interaction member and the balancing member may be arranged at the actuating element. The balancing member may be arranged opposite to the interaction member. The balancing member may prevent the actuating element from being tilted towards one side by the weight of the interaction member. Accordingly, the balancing member balances out the actuating element.

In one embodiment, the housing may comprise a window showing a counted number of the counting mechanism. Accordingly, a part of the toothed wheel may be visible through the window. In particular, one digit of the first toothed wheel, one digit of the second wheel and one digit of the third wheel may be visible through the window.

Further features and refinements become apparent from the following description of the exemplary embodiments in connection with the accompanying figures.

According to an alternate embodiment, an inhalation device for delivering at least one dose of a medicament is provided, comprising an alternate dose counting mechanism and a lock-out mechanism to prevent an operation of the inhalation device when a given number of doses, in particular a predefined number of doses, has been delivered. The operation configured to be prevented by the lock-out mechanism may be a dose delivery operation of the inhalation device. The alternate dose counting mechanism also has a first and a second state wherein the dose counting mechanism is configured to change from its first state to its second state by a user's suction airstream.

In particular, the alternate dose counting mechanism may comprise an interaction member coupled to an actuating element of the inhalation device. Further, the alternate dose counting mechanism may comprise a coupling member and a counting member. The coupling member may be configured such that a movement of the interaction member in a first direction, e.g. a proximal direction, is transferred to a movement of the counting member and to an incrementing or a decrementing of the counted number. In this context, the term “proximal end” may refer to the end of the inhalation device or the end of a part of the device furthest away from a dispensing end of the device. Accordingly, a movement in a proximal direction is a movement towards the proximal end. Further, the coupling member may be configured such that a movement of the interaction member in a second direction, e.g. a distal direction, is not transferred to a movement of the counting member and thereby the counted number is not altered. The term “distal end” may refer to the end of the inhalation device or a part of the inhalation device closest to the dispensing end. Accordingly, a movement in a distal direction is a movement towards the distal end.

Preferably, the operation is prevented by the lock-out mechanism permanently once the given number of doses has been delivered. For example, once the given number of doses has been delivered, a blocking element of the lock-out mechanism may engage with the interaction member, thereby preventing a further movement of the interaction member, in particular an axial movement of the interaction member. In particular, once the lock-out mechanism has prevented the operation, the operation may only be allowed again if the housing of the device is opened and the lock-out mechanism is released. Preferably, a medicament container needs to be changed or refilled in order to unlock the lock-out mechanism. In this case, the device may be configured to be a reusable device allowing for a container change. The lock-out mechanism may be configured such that it prevents an operation of the inhalation device when the container is empty, i.e. when the container dose not contain a medical substance.

Alternatively, the device may be configured such that once the lock-out mechanism has locked the device, i.e. prevented the operation, it is not possible to enable the operation again at all. In this case, the device may be configured to be disposed once the device has been locked.

When the user performs the operation, an audible or visible feedback may be provided to the user signaling to the user that the operation was successful. However, if the operation is prevented by the lock-out mechanism, the user may be alerted that the operation is not carried out successfully by the feedback not being provided. Accordingly, the missing feedback may be an indication to the user that a given number of doses has been delivered yet with a prior administering operation and the device is thus exhausted.

The given number of doses may, in particular, correspond to the total number of doses that can be provided by the inhalation device, in particular, the number of doses being provided by a medicament container. Accordingly, the lock-out mechanism prevents the operation when further doses can not be delivered, because the device is empty.

In one embodiment, the operation configured to be prevented by the lock-out mechanism is a dose delivery operation of the inhalation device. However, a dose setting operation may still be possible. In particular, the device may be configured such that an element, in particular an element of a drive mechanism of the device, has to be moved during the operation, in particular, the dose delivery operation. The lock-out mechanism may prevent a movement of said element and, thereby, prevent the dose delivery operation.

In one embodiment, the lock-out mechanism comprises a blocking element having a blocking position, wherein when the blocking element is in the blocking position the operation of the inhalation device is prevented.

In particular, the blocking element may prevent a movement of the element that has to be moved during the operation of the device, in particular during the dose delivery operation. In particular, the blocking element may prevent a movement of the element relative to a housing of the device. As an example, the blocking element may engage with the element, e.g. the blocking element may mechanically fix the element.

In one embodiment, the blocking element further has an unblocking position allowing the operation of the inhalation device unless the given number of doses has been delivered. In the unblocking position, the blocking element may not mechanically engage with the element that is configured to move in the operation, in particular the dose delivery operation. Thereby, the dose delivery operation may be allowed.

In one embodiment, the inhalation device is configured such that the blocking element changes from its unblocking position to its blocking position when the given number of doses has been delivered. In particular, the device may be configured such that the blocking element changes from its unblocking position to its blocking position when the given number of doses has been delivered and when further a cap has been screwed onto a housing of the inhalation device. Screwing the cap onto the housing may cause an update of the dose counting mechanism. When the dose counting mechanism has been updated and, thus, has counted the given number of doses, a movement of the blocking element to its blocking position may be caused. Accordingly, the device may be configured such that the blocking element moves into its blocking position when the given number of doses has been delivered and the dose counting mechanism has been updated.

In particular, when the given number of doses has been delivered, a path between the unblocking position and the blocking position of the blocking element may be cleared such that the blocking element is allowed to engage with the element that is moved during the operation, in particular during dose delivery. Said element may be the interaction member which is coupled to the actuating element. Accordingly, the blocking element prevents a movement of the interaction member, e.g. an axial movement in the distal direction, and further, as the interaction member is coupled to the actuating element, the blocking element also prevents a movement of the actuating element, e.g. an axial movement in the distal direction.

In one embodiment, the dose counting mechanism counts a number of doses, in particular the dose counting mechanism may increment or decrement a value corresponding to, e.g., the number of doses left in the device or the number of doses that have been delivered by the device.

The dose counting mechanism may provide information to a user about said number of doses. Therefore, the dose counting mechanism may increase the user-friendliness of the inhalation device.

In one embodiment, the lock-out mechanism may be configured to prevent an operation of the inhalation device when the number counted by the dose counting mechanism has a first predefined numerical value. This first predefined numerical value may correspond to the given number of doses. The lock-out mechanism may also prevent a further operation of the dose counting mechanism when the counted number has a first predefined numerical value. The operation of the dose counting mechanism prevented by the lock-out mechanism may be incrementing or decrementing the number.

In one embodiment, the dose counting mechanism may comprise a counting member. The counting member may comprise a wheel. Preferably, the counting member is movable, in particular rotatable. The counting member may have at least a first and a second configuration, in particular a first and a second orientation. Preferably, the counting member is configured to prevent a movement of the blocking element when the counting member is in its first configuration. Further, when the counting member is in its second configuration it may allow a movement of the blocking element. As an example, when the counting member is moved from its first configuration to its second configuration, a movement of the blocking member from the unblocking position to the blocking position may be allowed. Alternatively, the movement of the counting member from its first configuration to its second configuration may allow a movement of the blocking member which is not a movement into the blocking position. Accordingly, after the allowed movement, the blocking member may still be in an unblocking position.

The counting member may provide a path which allows a movement of the blocking element when the counting member is in its second configuration. The path element may comprise an opening provided in the counting member, in particular a hole through the counting member. Preferably, the path is provided in the counting member in a position different from the center of the counting member. Thereby, the position of the path alters when the counting member rotates about an axis extending through the center of the counting member.

The counting member may comprise digits. In particular, the counting member may comprise digits on its side face. The side face may be a surface facing away from a symmetry axis of the counting member.

In one embodiment, the blocking element is enabled to move to its blocking position when the path and the blocking element are aligned. In particular, the blocking element and the path may be aligned when the counting member is in its second configuration. Preferably, the counting mechanism is configured such that the path and the blocking element are aligned when the given number of doses has been delivered and when the counting mechanism has counted the given number. The path may allow a movement of the blocking element, in particular a movement enabling contacting the element which has to be moved during the operation to be prevented by the blocking element.

In one embodiment, the dose counting mechanism may comprise a second counting member. The second counting member may have a similar shape as the first counting member.

In particular, the second counting member may provide a wheel. Preferably, the second counting member is movable, in particular rotatable. The second counting member may have at least a first and a second configuration, in particular a first and a second orientation. Preferably, the second counting member is configured to prevent a movement of the blocking element when the second counting member is in its first configuration. Further, when the second counting member is in its second configuration it may allow a movement of the blocking element.

The second counting member may comprise a path which allows a movement of the blocking element when the second counting member is in its second configuration. The path may comprise an opening provided in the second counting member, in particular a hole through the second counting member. Preferably, the path is provided in the second counting member in a position different from the center of the counting member. Thereby, the position of the path alters when the second counting member rotates about an axis extending through the center of the second counting member.

The second counting member may comprise digits. In particular, the second counting member may comprise digits on its side face. The side face may be a surface facing away from a symmetry axis of the second counting member.

The inhalation device may be configured such that the blocking element is enabled to move to its blocking position only when all of the counting members are in their respective second configuration. In particular, the blocking element can only move to its blocking position when the path of each of the counting members is aligned with the blocking element.

In particular, the blocking element may gradually move to its blocking position by successive movements. A first movement may be carried out when the second counting member reaches its second configuration. In particular, the blocking element may then be aligned with the path of the second counting member. However, this movement may not cause an engagement of the blocking member and the element that is moved during the operation, thereby not preventing a further movement of the element. In particular, an engagement of the blocking member and the element that is moved during the operation may be prevented by the first counting member being in its first configuration. Accordingly, the blocking member may still be in an unblocking position.

A second movement may be carried out when the first counting member reaches its second configuration. In particular, the blocking element may then be aligned with the path of the first counting member. This movement may cause an engagement of the blocking member and the element that is moved during the operation, thereby preventing a further movement of the element that is moved during the operation. Accordingly, the blocking member is then in its blocking position.

In one embodiment, the dose counting mechanism may comprise an interaction member and a coupling member. The interaction member may be configured to interact with the coupling member when the interaction member is moved in a first direction. The first direction may be a proximal direction.

The interaction member may be moved in the first direction after a dose delivery operation has been completed. In particular, the interaction member may be moved in the first direction when a user performs an after-dose delivery operation, e.g. screws a cap onto a housing of the device.

The coupling member may be coupled to the counting members and, thereby, transmit a movement of the interaction member to the counting members such that the counted number is incremented or decremented. Alternatively, the coupling member may be provided by a counting member.

The coupling member may comprise a toothed wheel or a Geneva wheel, for example. A movement of the interaction member in a first direction may be transferred into a movement of the coupling member, in particular a rotational movement of the coupling member. Further, a movement of the coupling member may be transferred into a movement of at least one of the counting members.

In particular, the coupling member may be coupled to at least one of the counting members directly or by a gearing. The gearing may transfer a rotation of the coupling member by a first angle into a rotation of the counting member by a second angle.

The interaction member may be enabled to engage with the coupling member during at least parts of the movement of the interaction member in the first direction. The interaction member may not engage with the coupling member during its movement in the second direction.

The movement of the interaction member in the second direction may be a movement of the interaction member in a distal direction. This movement may correspond to a dose delivery operation.

The interaction member may be enabled to interact with the coupling element to change the counted number. The interaction member may preferably be enabled to rotate the coupling element by a predefined angle.

In one embodiment, the dose counting mechanism further comprises a guide track preventing an interaction of the interaction member and the coupling element when the interaction member is moved in a second direction.

In one embodiment, the inhalation device may comprise an actuating element being displaceable from a first position to a second position. The lock-out mechanism may be enabled to prevent a displacement of the actuating element from its first position to its second position when the given number of doses has been delivered. The actuating element may comprise or be the element that has to be moved during the dose delivery operation. The actuating element may be displaceable by a user's suction airstream. The interaction member may be provided by the actuating element. Preferably, the interaction member is an integral part of the actuating element or may be fixed to the actuating element. Accordingly, the interaction member is not enabled to move axially relative to the actuating element. Therefore, if the actuating element is moved axially in the proximal or distal direction, the interaction member follows said movement.

In one embodiment, the lock-out mechanism may further comprise a biasing member being coupled to the blocking element. The biasing member may exert a biasing force on the blocking element, in particular, when the blocking member is in the unblocking position. The biasing member may comprise a spring. The biasing member may be tensed when the blocking member is in the unblocking position of the lock-out mechanism. Further, the biasing member may be allowed to relax when the blocking element moves to its blocking position.

### BRIEF DESCRIPTION OF THE DRAWINGS

In one embodiment, the biasing member may be configured to move the blocking element from its unblocking position to its blocking position when the given number of doses has been delivered. In particular, the biasing member may move the blocking element into the paths of the at least one counting member and further into engagement with the interaction member.

FIG. 1 schematically shows a sectional side view of an inhalation device,

FIG. 2 schematically shows a sectional side view of a part of an inhalation device,

FIG. 3 schematically shows an interaction member and a toothed wheel in a first state of a dose counting mechanism,

FIG. 4 schematically shows an interaction member and a toothed wheel in a second state of a dose counting mechanism,

FIG. 5 schematically shows a sectional view of a part of the inhalation device,

FIG. 6 schematically shows a sectional side view of a part of an inhalation device according to an alternate embodiment,

FIG. 7 schematically shows parts of the inhalation device according to the alternate embodiment in an exploded view,

FIG. 8 schematically shows the interaction of an interaction member and a counter drive element in an inhalation device according to the alternate embodiment, and

FIG. 9 schematically shows a sectional side view of a part of the inhalation device according to the alternate embodiment in a locked-out configuration.

### DETAILED DESCRIPTION

Like elements, elements of the same kind and identically acting elements may be provided with the same reference numerals in the figures.

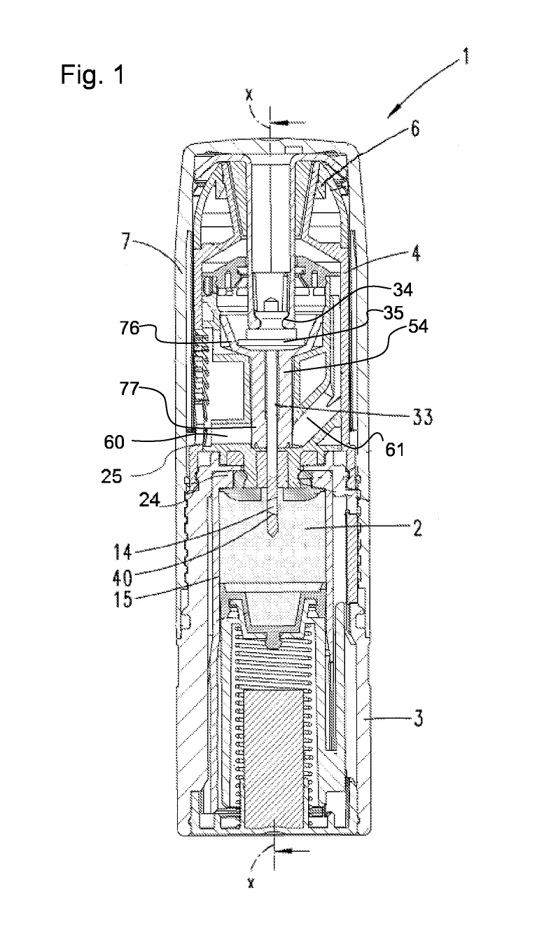

In FIG. 1, a sectional side view of an inhalation device 1 is shown.

The inhalation device 1 comprises a housing 3. The device 1 comprises an outer cylinder 4. The outer cylinder 4 is secured against axial movement with respect to the housing 3. The outer cylinder 4 is rotatable with respect to the housing 3.

The inhalation device 1 further comprises a mouthpiece 6. The inhalation device 1 comprises a cap 7. The cap 7 is used for covering the mouthpiece 6. The cap 7 may comprise a screw thread. The cap 7 may be rotatable with respect to the housing 3 for screwing the cap 7 onto the device 1 and for unscrewing the cap 7 from the device 1. The outer cylinder 4 is rotationally fixed to the cap 7. In particular, the outer cylinder 4 follows rotation of the cap 7 with respect to the housing 3. For the detailed description of the components of the inhalation device 1 and their mechanical cooperation it is referred to document WO 2009,065707 A1.

The device 1 comprises a storage chamber 15. The storage chamber 15 holds one dose, preferably a plurality of doses, of a medical substance 2. The substance 2 may be a powder.

The storage chamber 15 is terminated by a chamber sealing 24. The chamber sealing 24 is formed integrally with a top wall of the storage chamber 15. The device 1 further comprises a rotary part 25. The rotary part 25 is of substantially plate-like configuration and is connected in a rotationally fixed manner to the outer cylinder 4. Accordingly, the rotary part 25 follows rotation of the cap 7 and, hence, of the outer cylinder 4 about a main longitudinal axis x of the device 1 with respect to the storage chamber 15. However, the rotary body 25 is axially fixed relative to the housing 3.

The device 1 further comprises a metering rod 33. The metering rod 33 may be connected to the cap 7 by a snap fit element 34 when the cap 7 is engaged to the housing 3. The metering rod 33 is rotatably connected to the rotary part 25 by mechanical cooperation with the rotary part 25. Accordingly, the metering rod 33 follows rotational movement of the cap 7 and, hence, of the rotary part 25 about the main longitudinal axis x when the cap 7 is mounted onto the device 1 or demounted from the device 1.

When the cap 7 is engaged to the housing 3 the metering rod 33 travels axially in the proximal direction such that the most proximal part of the metering rod 33 comprising a metering chamber 40 enters the storage chamber 15. When the cap 7 is disengaged from the housing 3 the metering rod 33 travels axially in the distal direction such that the most proximal part of the metering rod 33 exits the storage chamber 15. In this context, “distal” may refer to the end of the inhalation device closest to the mouthpiece 6. Accordingly, “proximal” may refer to the end of the inhalation device furthest away from the mouthpiece 6.

In particular, the metering rod 33 is configured for functioning as a moving metering chamber 40 for a sub-quantity 14 of the substance 2 which is to be dispensed. The metering chamber 40 is formed in that end section of the metering rod 33 which projects into the substance 2.

The inhalation device 1 further comprises a flow path comprising a flow channel 60 and an intermediate channel portion 61.

The inhalation device 1 further comprises an actuating element 54. The actuating element 54 comprises a piston comprising tongues 77 and a piston head 76. The actuating element 54 has a first and a second position. The first position is more proximal than the second position. In the first position, the tongues 77 of the actuating element 54 block the flow path between the flow channel 60 and the intermediate channel portion 61. In its second position, the actuating element 54 is positioned further distal such that the tongues 77 do not block the flow path between the flow channel 60 and the intermediate channel portion 61.

The cap 7 is disengaged from the housing 3 by unscrewing the cap 7 from the housing 3. Accordingly, the cap 7 performs concurrently an axial movement in the distal direction and a rotational movement. During disengagement of the cap 7 from the housing 3, the rotational movement of the cap 7 is transferred into a rotation of the rotary part 25 around the longitudinal axis x. The rotation of the rotary part 25 is transferred into a rotation of the actuating element 54. Further, the concurrent axial and rotational movement of the cap 7 is transferred to the metering rod 33 concurrently performing an axial movement in the distal direction and a rotational movement around the longitudinal axis x. As the cap 7 approaches the end of the threaded connection to the housing 3, the snap fit element 34 is disengaged from the metering rod 33.

During disengagement of the cap 7 from the housing 3, the actuating element 54 is not moved axially relative to the housing 3. Accordingly, the actuating element 54 is in its first position before and after disengagement of the cap 7 from the housing 3.

When the cap 7 is fully disengaged from the housing 3, the metering chamber 40 is in a first condition. The first condition of the metering chamber 40 is defined by the tongues 77 of the actuating element 54 closing the metering chamber 40 such that the metering chamber 40 is not in contact with the flow path. Accordingly, when the actuating element 54 is in its first position and the cap 7 is disengaged from the housing 3, the metering chamber 40 is in its first condition.

In the first condition of the metering chamber 40, the tongues 77 of the actuating element 54 cover over the metering chamber 40 on each side. Accordingly, in this first condition, it is not possible for the sub-quantity 14 of substance to trickle out. Rather, the substance is held captive in the metering chamber 40.

After the cap 7 has been demounted, the user may trigger an inhalation operation by subjecting the device to a suction airstream, in the simplest case by the user breathing in. Air is sucked in via the mouthpiece 6, and this, in first instance, by virtue of the piston head 76 being subjected to the action of air, results in the actuating element 54 being displaced axially in the direction of the mouthpiece 6.

By virtue of the axially displaced actuating element 54, the tongues 77 are likewise displaced axially, in order to release the metering chamber 40. The metering chamber 40 is then in a second condition. The second condition of the metering chamber 40 is defined by the actuating element 54 being in its second position. In its second condition, the metering chamber 40 lies freely in a flow path between a flow channel 60 and an intermediate channel portion 61. The metering chamber is cleared out with air being sucked from the flow channel 60.

After the inhalation operation, the cap 7 may be engaged to the housing 3. During engagement of the cap 7 to the housing 3, the cap 7 is moved axially in the proximal direction and concurrently rotated around the longitudinal axis x. The snap fit element 34 engages to the metering rod 33 at the beginning of the threaded connection. Thereby the metering rod 33 is rotated and moved into the proximal direction when the cap 7 is engaged to the housing 3.

The metering rod 33 comprises a shield 35. The axial movement of the metering rod 33 during engagement of the cap 7 to the housing 3 is mechanically transferred to the actuating element 54 by the shield 35. In particular, the actuating element 54 is pushed from its second position to its first position. If the actuating element 54 is already in its first position, it is not moved axially during engagement of the cap 7 to the housing 3.

The inhalation device 1 further comprises a dose counting mechanism which, for clarity reasons, is not depicted in FIG. 1.

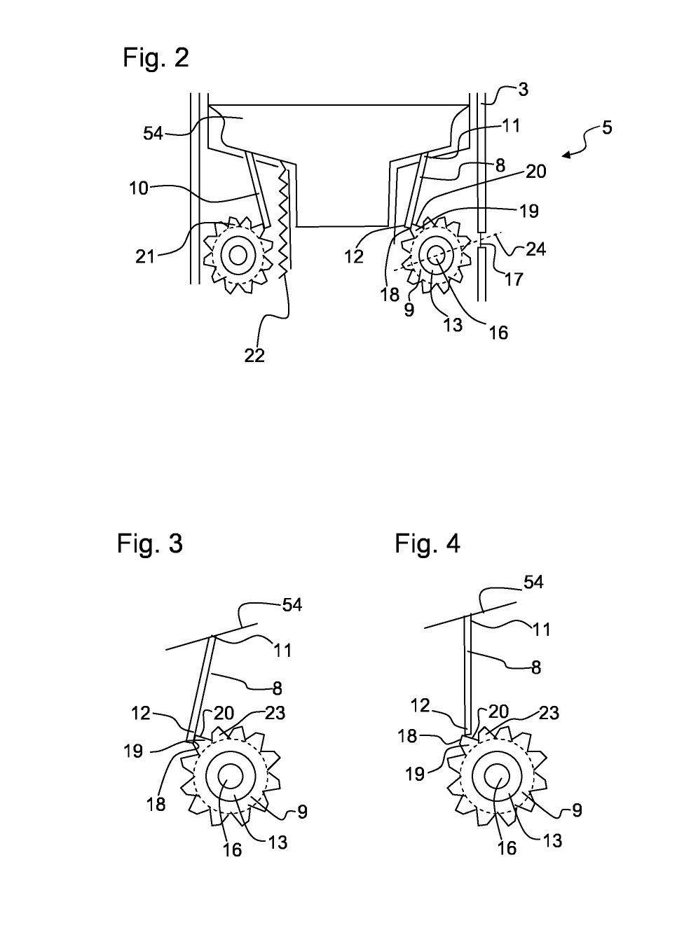

FIG. 2 schematically shows a sectional side view of a part of the inhalation device 1. In particular, the dose counting mechanism 5 is shown in FIG. 2. The dose counting mechanism 5 comprises an interaction member 8 and a counting member, e.g. a toothed wheel 9. Further, the dose counting mechanism 5 comprises a balancing member 10.

The interaction member 8 is coupled to the actuating element 54. The interaction member 8 may be an integral part of the actuating element 54. The interaction member 8 is a deflectable element. In particular, the interaction member 8 is a deflectable stick. One end 11 of the stick is connected to the actuating element 54. The other end of the stick forms a freely suspended tip 12.

The dose counting mechanism 5 comprises a first toothed wheel 9. The dose counting mechanism 5 may also comprise a second wheel 13 and optionally a third toothed wheel 16. The interaction member 8 is enabled to interact with the first toothed wheel 9. In particular, the interaction member 8 is enabled to cause rotations of the first wheel 9 by predetermined angles. The first toothed wheel 9 is coupled to the second and the third wheels 13, 16 such that, after a complete rotation of the first toothed wheel 9, the second wheel 13 is rotated by a predetermined angle. Further, after a complete rotation of the second wheel 13, the third wheel 16 is rotated by a predetermined angle.

Accordingly, the first toothed wheel 9 is a coupling member enabled to transfer a movement of the interaction member 8 into a movement of a first counting member. In this case, the first toothed wheel 9 is also the first counting member. Accordingly, coupling member and first counting member are provided by the same element, i.e. the toothed wheel 9.

The wheels 9, 13, 16 comprise digits. Further, the housing 3 of the inhalation device 1 may comprises a window 17. The window 17 is positioned such that one digit of each of the wheels 9, 13, 16 is visible through the window 17. A number is formed by the digits of the wheels 9, 13, 16 and the number is visible through the window 17. The counting mechanism 5 is configured such that, after removal of the cap 7, the number formed by the digits of the toothed wheels 9, 13, 16 corresponds to the numbers of doses left in the inhalation device 1.

Moreover, the dose counting mechanism 5 further comprises a balancing member 10. The balancing member 10 may be identical to the interaction member 8 regarding its shape, its material and its weight. In particular, the balancing member 10 may be a deflectable stick. Further, the dose counting mechanism 5 may comprise a dummy toothed wheel 21 for interaction with the balancing member 10. The balancing member 10 ensures that the actuating element 54 is in a balanced position as the balancing member 10 presents a counterweight to the interaction member 8. In particular, the balancing member 10 ensures that the actuating element 54 is not tilted to the side of the interaction member 8.

Furthermore, the dose counting mechanism 5 may comprise a spring 22. The spring 22 is enabled to exert a force on the actuating element 54. The actuating element 54 is moved from its first position to its second position if a user exerts a predetermined force by a suction airstream. However, the interaction member 8 interacting with the first toothed wheel 9 and the balancing member 10 interacting with the dummy toothed wheel 21 may increase the force required to move the actuating element 54 to its second position. The spring 22 allows adjusting the force required to move the actuating element 54 to its second position. In particular, the spring 22 applies a force to the actuating element 54 in the direction of the mouthpiece 6, thereby decreasing the force necessary to move the actuating element 54 to its second position. Accordingly, the spring 22 compensates the effect of the interaction element 8 regarding the force required to move the actuating element 54.

FIG. 3 shows the interaction member 8 and the counting member in a first state of the dose counting mechanism 5. In particular, the interaction member 8 is deflected by a tooth 19 of the first toothed wheel 9. FIG. 4 shows the interaction member 8 and the counting member in a second state of the dose counting mechanism 5. In this state, the interaction member 8 is not deflected. The interaction member 8 abuts a side face 20 of the tooth 19 of the first toothed wheel 9.

In FIG. 3, the actuating element 54 is in a first position. The first position of the actuating element 54 corresponds to the first condition of the metering chamber 40. This condition is realized when the cap 7 is disengaged from the housing 3 and a dose delivery has not yet been performed. Accordingly, in the first position of the actuating element 54, the tongues 77 of the actuating element 54 cover over the metering chamber 40 on each side and the sub-quantity 14 of the substance 2 is held reliably captive in the metering chamber 40.

When the actuating element 54 is in its first position, the interaction member 8 is deflected. The freely suspended tip 12 of the interaction member 8 abuts a top face 18 of one tooth 19 of the first toothed wheel 9 and is thereby deflected by the tooth 19. The top face 18 of the tooth 19 is a surface that is inclined relative to the longitudinal axis x of the device.

When the dose counting mechanism 5 is in its first state and the interaction member 8 is accordingly in its first position, the interaction member 8 can only be moved in a distal direction. Further, in its first position, the interaction member 8 can not exert a torque on the first toothed wheel 9. Accordingly, when the dose counting mechanism 5 is in its first state and the interaction member 8 is accordingly in its first position, the first toothed wheel 9 can not be rotated by the interaction member 8 and, thereby, the counted number can not be altered.

Further, in this state, the counting mechanism 5 is in its deactivated state.

By a user's suction airstream the actuating element 54 is moved from its first position to a second position. The second position of the actuating element 54 is shown in FIG. 4. In this position, the tongues 77 are likewise displaced axially, in order to release the metering chamber 40. The metering chamber 40 is cleared out with air being sucked from the flow channel 60.

As the interaction member 8 is engaged to the actuating element 54, it is moved axially together with the actuating element 54. Thereby, the state of the dose counting mechanism 5 is altered from the first state to the second state. In particular, the interaction member 8 slides along the tooth 19 of the first toothed wheel 9. In the second state, the interaction member 8 abuts a side face 20 of the tooth 19. The side face 20 of the tooth 19 is a surface that is perpendicular to the longitudinal axis x of the inhalation device 1 and that faces towards the mouthpiece 6 when the dose counting mechanism is in its activated state. In the activated state, the dose counting mechanism 5 is ready to increment the counted number of the toothed wheels 9, 13, 16.

When the dose counting mechanism 5 is in its second state and the interaction member 8 is accordingly in its second position, the interaction member 8 can only be moved in a proximal direction. Further, in its second position, the interaction member 8 can exert a torque on the first toothed wheel 9 when the interaction member 8 is now moved in a proximal direction. Accordingly, when the dose counting mechanism 5 is in its second state and the interaction member 8 is accordingly in its second position, the first toothed wheel 9 can be rotated by the interaction member 8 and, thereby, the counted number can be altered.

However, if in this state the user performs a second suction at the mouthpiece 6, no dose of the substance 2 is delivered as the metering chamber 40 is empty. The second suction also does not move the actuating element 54. Thereby, the state of the dose counting mechanism 5 is not altered by the second suction.

After dispensing the sub-quantity 14 of the medical substance 2, the user may screw the cap 7 to the housing 3. By engaging the cap 7 to the housing 3, the actuating element 54 is moved axially from its second position to its first position. Thereby, the interaction member 8 is also moved axially in a direction away from the mouthpiece 6.

Due to the engagement of the cap 7 to the housing 3, and as the actuating element 54 is moved to its first position, a force in a proximal direction is applied to the interaction member 8. The interaction member 8 abuts the tooth 19 of the toothed wheel 9 and transmits this force to the side face 20 of the tooth 19. Thereby, the interaction member 8 pushes the tooth 19 of the first toothed wheel 9. Thereby, the toothed wheel 19 is rotated by a predetermined angle.

Accordingly, the counting of the dose counting mechanism 5 is incremented or decremented by one unit. Further, the dose counting mechanism 5 is thereby altered from the second state to the first state. In the first state, the tip 12 of the interaction member 8 now abuts the top face 18 of a second tooth 23 of the toothed wheel 9 that is adjacent to the tooth 19 that had been abutted in the previous deactivated state of the counting mechanism 5.

Furthermore, if the user has disengaged the cap 7 from the housing 3 without a dose being delivered afterwards, the actuating element 54 remains in its first position. Further, the interaction member 8 is coupled to the actuating element 54. Therefore, if the user has disengaged the cap 7 from the housing 3 without a dose being delivered afterwards, the interaction member 8 also remains in its first position, i.e. the freely suspended tip 12 of the interaction member 8 abuts a top face 18 of one tooth 19 of the first toothed wheel 9 and is thereby deflected by the tooth 19 (see FIG. 3). If the user now engages the cap 7 to the housing 3, this engagement does not move the actuating element 54 and, therefore, does not move the interaction member 8. Accordingly, in this case, the dose counting mechanism 5 remains in its first state. Therefore, a disengagement and an engagement of the cap 7 to the housing 3 without a dose being delivered in between does not move the actuating element 54 and therefore does not increment or decrement the counted number counted by the dose counting mechanism 5.

As the dose counting mechanism 5 changes the counted number only when the cap 7 is screwed to the housing 3 and after a dose delivery has been performed previously, the engagement of the cap 7 to the housing 3 corresponds to updating the counted number shown by the dose counting mechanism 5. Therefore, when the cap 7 is removed from the housing 3, the dose counting mechanism 5 always shows the correct number of doses left in the device 1.

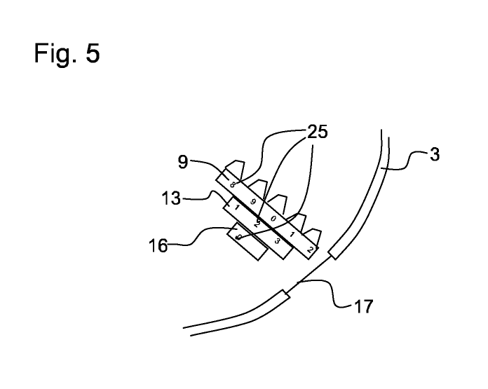

FIG. 5 shows a sectional view taken along the dotted line 24 of FIG. 2. Each of the three wheels 9, 13, 16 comprises digits 25.

A user can see one digit 25 of each of the wheels 9, 13, 16 through the window 17 of the housing 3.

The wheels 9, 13, 16 may display the number of doses left in the device 1. Alternatively, the wheels 9, 13, 16 may display the number of doses delivered by the device 1. The first toothed wheel 9 corresponds to the unit digit of the doses, the second wheel 13 corresponds to the tens digit of the doses left and the third wheel 16 corresponds to the hundreds digit. Each of the wheels 9, 13, 16 comprises digits 25 such that by a combination of the digits 25 on the wheels 9, 13, 16 the number of doses left in the inhalation device 1 or delivered by the inhalation device 1 is shown. However, each of the wheels 9, 13, 16 needs to show only the digits corresponding to possible values. Assuming that there are a total number of 200 doses, the third wheel 16 may only comprise the digits 0, 1 and 2. In this case, the third wheel 16 may be smaller than the first or second wheel 9, 13.

Thereby, the size of the counting mechanism 5 may be adjusted to the curved shape of the outer housing 3.

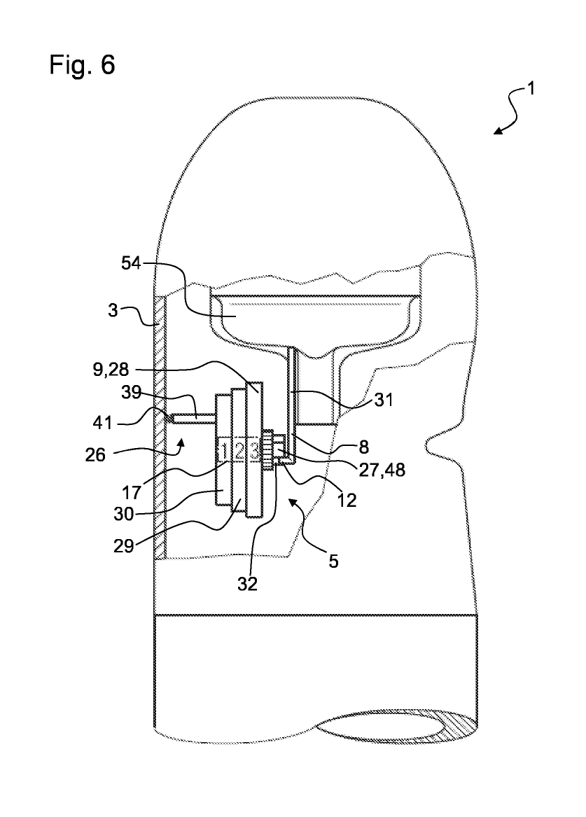

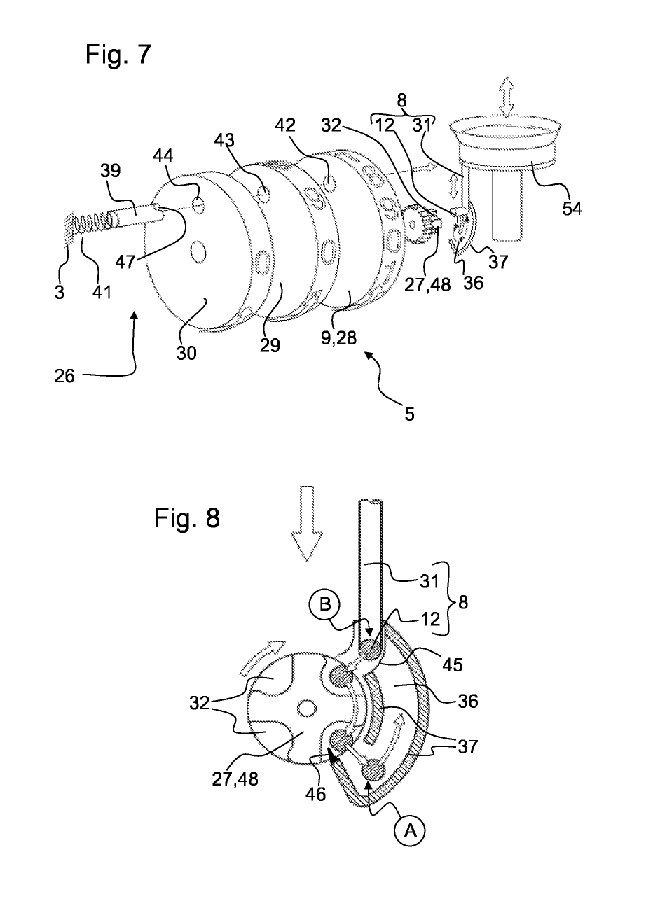

Furthermore, FIG. 6 schematically shows a sectional side view of a part of the inhalation device 1 according to an alternate embodiment. Further, FIG. 7 shows an exploded view of parts of the inhalation device 1, in particular of the counting mechanism 5 according to the alternate embodiment.

The dose counting mechanism 5 according to the alternate embodiment also has a first state and a second state, wherein the counting mechanism 5 is configured to change from the first state to the second state by a user's suction airstream.

The dose counting mechanism 5 comprises the interaction member 8, a coupling element 27, a first counting member 28, a second counting member 29 and a third counting member 30. In particular, the first counting member 28 may comprise a first counting wheel. The second counting member 29 may comprise a second counting wheel and the third counting member 30 may comprise a third counting wheel.

The coupling element 27 is coupled to the first counting member 28. The first counting member 28 is disk-shaped and has digits on its side face. The surface normal to the side face is perpendicular to a symmetry axis of the first counting member 28. The first counting member 28 is enabled to rotate around the symmetry axis.

The interaction member 8 is provided by the actuating element 54. The interaction member 8 may be an integral part of the actuating element 54. The interaction member 8 is a deflectable element. In particular, the interaction member 8 is configured as a deflectable stick. The interaction member 8 comprises a main part 31 and a freely suspended tip 12. One end of the main part 31 is connected to the actuating element 54. At the other end of the main part 54, the freely suspended tip 12 is located, wherein the tip 12 extends at an angle relative to the main part 31. In particular, the freely suspended tip 12 forms a rectangular angle with respect to the main part 31 of the interaction member 8.

The coupling element 27 is configured to transfer a movement of the interaction member 8 into a movement of at least one of the counting members 28, 29, 30. In particular, the coupling element 27 may transfer a movement of the interaction member 8 in the proximal direction into a rotational movement of the first counting member 28. Further, the counting mechanism may be configured such that a movement of the interaction member 8 in the distal direction is not transferred into a movement of the coupling element 27 and the first counting member 28.

The coupling element 27 and the first counting member 28 are coupled by a first gearing which is not shown in FIGS. 6 and 7. Accordingly, a rotation of the coupling element 27 is transferred into a rotation of the first counting wheel 28. Preferably, the first gearing is designed such that a rotation of the coupling element 27 by an angle is transferred into a rotation of the first counting member 28 by a smaller angle. Preferably, the gearing is configured such that ten dose delivery operations correspond to a complete rotation of the first dose counting member 28 by 360°.

Moreover, the counting mechanism 5 comprises a second and a third counting member 29, 30. The second and the third counting members 29, 30 are also disk-shaped, having digits on their side faces. The second counting member 29 is coupled to the first counting member 28, e.g. by a second gearing such that a complete rotation of the first counting member 28 by 360° is transferred into a rotation of the second counting member 29 by a predefined angle. The predefined angle may be chosen such that after 100 delivered doses the second counting member is rotated by 360°.

Further, the third counting member 30 is coupled to the second counting member 29 by a third gearing such that a complete rotation of the second counting member 29 is transferred into a rotation of the third counting member 30 by a predefined angle.

In the embodiment shown in FIGS. 6-9, the coupling element 27 is a Geneva wheel 48. In an alternative embodiment which is not shown in the Figures the coupling element 27 and the first counting member 28 may be formed by a single element fulfilling both functions.

Further, the Geneva wheel 48 comprises a plurality of coupling areas. In particular, the Geneva wheel 48 comprises a plurality of receptacles 32. In particular, in the embodiment shown in FIGS. 6 to 9, the Geneva wheel 48 comprises four receptacles 32. The receptacles 32 are positioned at equally spaced circumferential positions at the Geneva wheel 48. Each receptacle 32 is sized and positioned to receive the freely suspended tip 12 of the interaction member 8 when the Geneva wheel 48 and the suspended tip 12 are in predefined specific states.

In particular, the freely suspended tip 12 of the interaction member 8 is configured to engage into a receptacle 32 of the Geneva wheel 48 and to rotate the Geneva wheel 48 by a predetermined angle, e.g. by an angle of 90°.

The Geneva wheel 48 is designed such that the freely suspended tip 12 engages with the Geneva wheel 48 when the interaction member 8 is moved from a distal position in a proximal direction. The distal position is the second position of the interaction member 8, marked as “B” in FIG. 8. The proximal position is the first position of the interaction member 8, marked as “A” in FIG. 8.

For this purpose, an entry guide lip 45 may be provided which initiates the engagement of the freely suspended tip 12 with the Geneva wheel 48 at the beginning of the movement of the interaction member 8 in the proximal direction.

FIG. 8 schematically shows the interaction of the interaction member 8 and the Geneva wheel 48. When the actuating element 54 is in its first position also the interaction member 8 is in a first position. The first position of the interaction member 8 is indicated as position “A” in FIG. 8. The first position of the actuating element 54 corresponds to the first condition of the metering chamber 40. This condition is present when the cap 7 has been disengaged from the housing 3 and a dose delivery has not yet been performed. Accordingly, in the first position of the actuating element 54, the tongues 77 of the actuating element 54 cover over the metering chamber 40 on each side and the sub-quantity 14 of the substance 2 is held reliably captive in the metering chamber 40.

Further, the interaction member 8 and the actuating element 54 being in their respective first positions A correspond to the dose counting mechanism 5 being in its first state. When the interaction member 8 is in its first position A, it is disengaged from the coupling element 27. In particular, in its first position A, the interaction member 8 is positioned in a guide track 36 adjacent to the coupling element 27. The guide track 36 is configured such that an interaction of the interaction member 8 and the coupling element 27 prevented when the interaction member 8 is in its first position A.

When the actuating element 54 is in its first position, a user's suction airstream initiates a dose delivery. The user's suction airstream moves the actuating element 54 from its first position to its second position. In this position, the tongues 77 are likewise displaced axially, in order to release the metering chamber 40. The metering chamber 40 is cleared out with air being sucked from the flow channel 60. When the actuating element 54 is moved from its first position to the second position, the interaction member 8 is also moved from its first position to its second position. The second position of the interaction member 8 is indicated as position “B” in FIG. 8. The interaction member 8 is moved from its first position A to its second position B along the guide track 36. The guide track 36 runs adjacent to the coupling member 27 such that during its movement from its first position A to its second position B the interaction member 8 does not contact the coupling member 27 and, therefore, dose not interact with the coupling member 27. Accordingly, when the interaction member 8 is moved from its first position A to its second position B, the counted number of the dose counting mechanism 5 is not altered as the coupling member 27 and, thereby, the counting members 28, 29, 30 are not moved.

The dose counting mechanism 5 is configured such that an update of the counted number, i.e. a possible incrementing or decrementing of the counted number, is carried out when a user engages the cap 7 to housing 3. When the actuating element 54 and the interaction member 8 are in their respective first positions which corresponds to the dose counting mechanism 5 being in its first state, and further, the cap 7 is engaged to the housing, the actuating element 54 and the interaction member 8 are not moved. Accordingly, the interaction member 8 does not interact with the coupling member 27 and the counted number of the dose counting mechanism 5 is not altered.

Moreover, when the dose counting mechanism 5 is in its first state, a user's suction airstream changes the dose counting mechanism 5 to its second state. In particular, the interaction member 8 and the actuating element 54 being in their respective second positions B correspond to the dose counting mechanism 5 being in its second state. When the dose counting mechanism 5 is in its second state, the counted number is updated, i.e. incremented or decremented, when the user engages the cap 7 to the housing 3. When the actuating element 54 and the interaction member 8 are in their respective second positions which corresponds to the dose counting mechanism 5 being in its second state, and further, the cap 7 is engaged to the housing, the actuating element 54 and the interaction member 8 are moved in the proximal direction. Thereby, the interaction member 8 engages with the coupling member 27. The axial movement of the interaction member 8 in the proximal direction is transferred into a rotation of the coupling member 27. This rotation is further transferred into a movement of at least one of the counting members 28, 29, 30, thereby incrementing or decrementing the counted number.

Moreover, the counting mechanism 5 comprises the guide track 36. The entry guide lip 45 is provided on the guide track 36.

Further, the guide track 36 comprises two guide rails 37. The guide track 36 is located at the side of the Geneva wheel 48. When the interaction member 8 moves from its first to its second position, the freely suspended tip 12 slides along the guide track 36 guided by the guide rails 37. The guide rails 36 allow for moving the freely suspended tip 12 along the side of the Geneva wheel 27 in a distal direction, in particular form the first position to the second position, without that the tip 12 interacts with the Geneva wheel 48. Instead, the freely suspended tip 12 is guided through the guide track 36 between the two guide rails 37 and thereby does not contact the Geneva wheel 48. The main part 31 of the interaction member 8 is moved in a plane that is offset to the guide rails 37 and, thereby, does not contact the guide rails 37.

When a dose delivery has been performed and the actuating element 54 and the interaction member 8 are in their respective second positions, the user may perform a second suction at the mouthpiece 6. However, in this case, no dose of the substance 2 is delivered as the metering chamber 40 is empty. The second suction also does not move the actuating element 54, as the actuating element 54 remains in its second position after a dose delivery until the cap 7 is engaged to the housing 3. Thereby, the interaction member 8 is not moved and, therefore, the state of the dose counting mechanism 5 is not altered by the second suction.

After dispensing the sub-quantity 14 of the medical substance 2, the interaction member 8 may be moved in the proximal direction when a user performs an after-dose delivery operation, e.g. the user may screw the cap 7 to the housing 3.

When the cap 7 is screwed on the housing 3, the actuating element 54 is moved into the proximal direction from its second position to its first position. As the interaction member 8 is coupled to the actuating element 54, the interaction member 8 is also moved in the proximal direction from its second position to its first position when the cap 7 is screwed onto the housing. Thereby, the freely suspended tip 12 of the interaction member 8 engages the Geneva wheel 48 and rotates the Geneva wheel 48 by 90°. This engagement is initiated by the entry guide lip 45 provided on the guide rail 37.

The rotation of the Geneva wheel 48 is transferred into a decrementing of the counted number of the counting mechanism 5. Accordingly, the movement of interaction member 5 from its second position B to its first position A causes the decrementing of the counted number of the counting mechanism. This corresponds to the counting mechanism changing from its second state to its first state, thereby decrementing the counted number.

However, near the end of the proximal movement of the interaction member 8, the freely suspended tip 12 disengages from the receptacle 32 of the Geneva wheel 48 and slides into the guide track 36. The guide track 36 may comprise a guide feature, e.g. an exit guide lip 46 that disengages the tip 12 from the receptacle 32. The exit guide lip 46 may be flexible such that it is bendable allowing for a rotation of the Geneva wheel 48. At the same time, it is stiff enough to allow for the tip 12 to slide along the exit guide lip 46 out of the receptacle 32 of the Geneva wheel 27 and into the guide track 36.

Alternatively, the interaction member 8 may disengage from the receptacle 32 and engage into a proximal opening of the guide track 36 due to a resilient force. During its proximal movement the interaction member 8 slides along the surface of one of the guide rails 37, in particular the surface facing away from the guide track 36. Near the end of the proximal movement, the interaction member 8 is enabled to swing into the proximal opening of the guide track 36. Accordingly, the exit guide lip 46 is optional. Furthermore, also the entry guide lip 45 may be optional.

Accordingly, once the cap 7 is screwed on the housing 3, the interaction member 8 is moved in a proximal direction and the counting mechanism 5 is thereby updated. In case, the cap 7 is unscrewed from the housing 3, but no dose delivery is performed, the actuating element 54 remains in its first position and, thereby, the interaction member 8 remains in its first position. Accordingly, the interaction member 8 is not moved from its second position to its first position when the cap 7 is screwed back onto the housing 3. Accordingly, the Geneva wheel 48 is not rotated and the counted number of the counting mechanism 5 is not altered.

As can be seen in FIG. 7, the first, the second and the third counting members 28, 29, 30 comprise digits. Further, the housing 3 comprises a window 17. The window 17 is arranged such that one digit of each of the counting member 28, 29, 30 is visible. The digits of the counting members 28, 29, 30 visible through the window 17 form a counted number which is decremented by the counting mechanism 5. The counted number corresponds to the numbers of doses left in the device 1. The digit of the first counting member 28 visible in the window 17 may correspond to the units digit of the number of left doses, the digit of the second counting member 29 visible in the window 17 may correspond to the tens digit and the digit of the third counting member 30 visible in the window 17 may correspond to the hundreds digit.

In an alternate embodiment, the counted number visible in the window 17 may correspond to the number of doses that have been delivered by the device 1. Accordingly, in this embodiment, the counted number is incremented by the counting mechanism 5.

Moreover, the dose counting mechanism 5 may further comprise a balancing member not shown in FIGS. 6 and 7. The balancing member may be identical to the interaction member 8 regarding its shape, its material and its weight. In particular, the balancing member may be a deflectable stick. The balancing member may be coupled to the actuating element 54. Preferably, the balancing member is arranged at the actuating element 54 opposite to the interaction member 8. The balancing member ensures that the actuating element 54 is in a balanced position by providing a counterweight to the interaction member 8. In particular, the balancing member ensures that the actuating element 54 is not tilted towards the side of the interaction member. Further, the dose counting mechanism 5 may comprise a dummy Geneva wheel for interaction with the balancing member.

Furthermore, the inhalation device 1 comprises a lock-out mechanism 26. The lock-out mechanism 26 comprises a blocking element 39. The blocking element 39 may be a lock-out pin. The blocking element 39 is coupled to the housing 3 by a biasing member 41, e.g. by a spring. The blocking element 39 has a blocking position and an unblocking position.

In particular, FIG. 6 shows the lock-out mechanism 26 in a configuration wherein the blocking element 39 is in its unblocking position. The blocking element 39 is arranged between the housing 3 and the third counting member 30 of the dose counting mechanism 5 and does not interact with the interaction member 8.

The lock-out mechanism 26 may prevent an operation of the inhalation device 1 when a given number of doses has been delivered. The operation configured to be prevented by the lock-out mechanism 26 may be e.g. a dose delivery operation of the inhalation device 1.

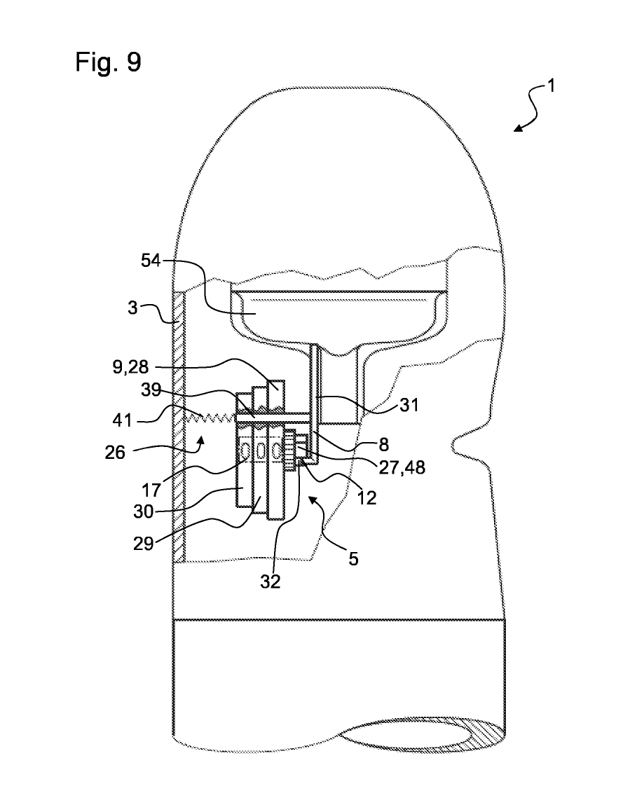

Each of the counting members 28, 29, 30 may have at least a first and a second configuration, in particular a first and a second orientation. Preferably, the counting members 28, 29, 30 are configured to prevent a movement of the blocking element 39 when the counting members 28, 29, 30 are in their respective first configuration (see FIG. 6). Further, when the counting members 28, 29, 30 are in their respective second configuration it may allow a movement of the blocking element 39 (see FIGS. 6 and 9).

As shown in FIG. 7, each of the counting members 28, 29, 30 comprises a path 42, 43, 44, e.g. formed by a hole through the counting member 28, 29, 30. The blocking element 39 is arranged such that it is enabled to move into the path 42, 43, 44 of the respective first, second or third counting member 28, 29, 30 when the paths 42, 43, 44 are aligned with the blocking element. The blocking member 39 may be enabled to move into the first, the second and the third coupling member successively. The blocking member 39 may be enabled to move to its blocking position by a movement comprising multiple sub-movements that may be separated in time. In particular, in a first sub-movement the blocking member 39 may move into the path 44 of the third counting member 30 and may be blocked from a further movement by the second counting member 29. In a second sub-movement the blocking member 39 may move into the path 43 of the second counting member 29 and may be blocked from a further movement by the first counting member 28. In a third sub-movement the blocking member 39 may move into the path 42 of the first counting member 28 and into engagement with the interaction member 8.

In particular, the blocking element 39 is arranged such that it is enabled to move into engagement with first path 44 of the third counting member 30 when the digit of the third counting member 30 visible through the window 17 equals zero. The blocking element 39 is movable into the path 44 of the third counting wheel 30 by a force exerted by the biasing member 41.

For example, the inhalation device 1 may comprise 150 doses of a medical substance 2. Accordingly, in its initial configuration the counting members 28, 29, 30 of the dose counting mechanism 5 show the number “150” in the window 17. Once 51 doses have been delivered, the number “099” is shown in the window 17. Now the blocking element 39 is aligned with the path 44 of the third counting member 30 and is pushed by the biasing member 41 into the path 44.

However, when 51 doses have been delivered, the second counting member 29 does not show a zero in the window 17 in this configuration. Accordingly, the blocking element 39 is not aligned with the path 43 of the second counting member 29. Therefore, the blocking element 39 is prevented from moving into engagement with the path 43 of the second counting member 29. Accordingly, the blocking element 39 is still in an unblocking position as it dose not prevent an operation of the interaction member 8 and of the actuating element 54.

Moreover, when delivering further doses, the second counting member 29 is gradually rotated. When the second counting member 29 shows a zero in the window 17, the path 43 of the second counting member 29 is aligned with the blocking element 39. Accordingly, the blocking element 39 now is enabled to move into engagement with the path 43 of the second counting wheel 29. In particular, the blocking element 39 is pushed into the path 43 of the second counting member 29 by the biasing member 41.

When the last dose is delivered and the cap 7 has been engaged to the housing 3, the path 42 of the first counting member 28 is aligned with the blocking element 39. In this case, the digit of the first counting member 28 visible in the window 17 equals 0. Accordingly, “000” is visible in window 17. The blocking element 39 now engages the path 42 of the first counting member 28. The paths 42, 43, 44 of all counting members 28, 29, 30 are aligned to the blocking element 39.

Further, the blocking element 39 is now enabled to engage the interaction member 8. Thereby, the blocking element 39 prevents the interaction member 8 from being moved axially. Accordingly, a dose delivery operation is not possible anymore.

FIG. 9 shows the inhalation device 1 in a configuration when all available doses have been delivered. The counting members 28, 29, 30 have been rotated such that “000” is visible in the window 17 and the blocking element 39 is aligned with the paths of all counting members 28, 29, 30. Accordingly, the biasing member 41 has pushed the blocking element 39 into the path 42 of the first counting member 28. By the force exerted by the biasing member 41, the blocking element 39 is moved towards the interaction member 8 and engages with the interaction member 8 of the dose counting mechanism 5. The blocking element 39 is designed such that it prevents a further axial movement of the interaction member 8 once it is engaged with the interaction member 8. Accordingly, the blocking element 39 is in its blocking position.

In the blocking position, the blocking element 39 may be prevented from a movement relative to the housing of the device by its engagement with the first counting member 28. In particular, one end of the blocking element 39 may be engaged to the interaction member 8 and the other end of the blocking element 39 may be located in the path 42 and, thereby, may be engaged with the first counting member 28 such that the blocking element 39 is locked against a movement at least in distal direction. In particular, the first counting member 28 may be configured such that a rotation of the counting member 28 in a rotational direction opposite the rotational direction during updating the counted number is prevented. Thereby, the blocking element 39 locks the interaction member 8 against a movement at least in distal direction.

The blocking element 39 may engage with the interaction member 8 at the connection point of the main part 31 and the tip 12. In particular, the blocking element may engage with the interaction member 8 at a point slightly more distal than the tip 12.

In particular, the front face of the blocking element 39 facing towards the interaction member has an inwardly curved surface 47 which is adapted to the surface of the interaction member 8. Thereby, the blocking element 39 fixes the interaction member 8 such that it cannot be moved axially anymore. The interaction member 8 is locked in its first position, which is marked as “A” in FIG. 8. The interaction member 8 is prevented from a movement in a distal direction by the engagement of the blocking element 39.

Additionally or alternatively, the interaction member 8 may comprise a recess. The blocking element 39 may be enabled to engage into the recess and, thereby, to lock the interaction member 8 such that the interaction member 8 can not move axially anymore.

By axially fixing the interaction member 8, the blocking element 39 prevents a further movement of the actuating element 54. In particular, it is no longer possible to move the actuating element 54 axially in a distal direction. Thereby, a dose delivery of the inhalation device 1 is prevented.

When the actuating element 54 is moved from its first position to its second position, an audible and visible feedback is provided to the user. In particular, when the actuating element 54 is moved into its second position, the piston head 76 (see FIG. 1) may engage into a corresponding projection, thereby providing an audible feedback. Moreover, the housing 3 of the inhalation device 1 may comprise a small opening, allowing the user to see if the actuating element is in its first or its second position as the actuating element blocks the opening in its first position and, further, does not block the opening in its second position.

However, if a user performs a suction at the mouthpiece 6 after the blocking element 39 has engaged the interaction member 8, no audible or visible feedback is provided to the user as the actuating element 54 is not moved. Thereby, the user is informed that no dose has been delivered. Accordingly, the lock-out mechanism 26 alerts the user that the inhalation device 1 does not provide further doses of the medical substance 2.

Once the blocking element 39 locks the interaction member 8, a dose delivery of the inhalation device 1 is permanently prevented. It is not possible to unlock the blocking element 39 from the interaction member 8 unless the housing 3 of the device 1 is opened. When the blocking element 39 locks the interaction member 8, a user may dispose the inhalation device 1. Alternatively, a user or a trained person may refill new doses of a medical substance 2 or change a container comprising the medical substance 2 and thereafter unlock the lock-out mechanism 26.

Furthermore, the dose counting mechanism 5 may comprise a second spring which is not shown in FIGS. 6 to 9. The second spring is enabled to exert a force on the actuating element 54 in a distal direction. The actuating element 54 is moved from its first position to its second position if a user exerts a predetermined force by a suction airstream. However, the interaction member 8 interacting with the counter drive element and the balancing member interacting with the dummy counter drive element may increase the force required to move the actuating element 54 to its second position. The second spring allows adjusting the force required to move the actuating element 54 to its second position. In particular, the spring applies a force to the actuating element 54 in the direction of the mouthpiece 6, thereby decreasing the force necessary to move the actuating element 54 to its second position. Accordingly, the second spring compensates the effect of the interaction element 8 regarding the force required to move the actuating element 54.

Moreover, the features that have been described with respect to the alternate embodiment may be combined with the first embodiment and, vice versa, the features that have been described with respect to the first embodiment may be combined with the alternate embodiment. For example, the embodiment shown in FIG. 1-5 may further comprise a lock-out mechanism 26 as disclosed with respect to the alternate embodiment. In particular, the lock-out mechanism may prevent a further operation of the inhalation device after a predefined number of doses has been delivered.

Moreover, the coupling member 27 being formed by the same element as the first counting member in the first embodiment may be modified to be an element separate from the first counting member as disclosed with respect to the alternate embodiment. In particular, the first embodiment may additionally comprise a coupling member 27 comprising a Geneva wheel 48.

The term “medical substance”, as used herein, may mean a pharmaceutical formulation containing at least one pharmaceutically active compound, for example for the treatment of obstructive airway or lung diseases such as asthma or chronic obstructive pulmonary disease (COPD), allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, or a human insulin analogue or derivative, a glucagon-like peptide (GLP-1) or an analogue or derivative thereof, or exendin-3 or exendin-4 or an analogue or derivative of exendin-3 or exendin-4;

an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol)); a glucocorticoid (e.g. Beclometasone, Budesonide, Ciclesonide, Fluticasone, Mometasone, Flunisolide, Betamethasone, Triamcinolone);

an anticholinergic agent or muscarinic antagonist (e.g. Ipratropium bromide, Oxitropium bromide, Tiotropium bromide);

a mast cell stabilizer (e.g. Cromoglicate, Nedocromil);

a xanthine derivative (e.g. Doxofylline, Enprofylline, Theobromine, Theophylline, Aminophylline, Choline theophyllinate);

an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);

or a combination of any two, three or more of the above-mentioned compound classes or compounds (e.g. Budesonide/Formoterol, Fluticasone/Salmeterol, Ipratropium bromide/Salbutamol, Mometasone/Formoterol);

or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

1-12. (canceled)

13. Inhalation device, comprising a dose counting mechanism and an actuating element, wherein the actuating element is configured to be moved during a dose delivery operation of the inhalation device, wherein the counting mechanism has a first state and a second state and wherein the counting mechanism is configured to change from the first state to the second state by a user's suction airstream, wherein the dose counting mechanism comprises an interaction member being enabled to operate a counting function of the dose counting mechanism, wherein the interaction member is an integral part of the actuating element of the inhalation device or may be fixed to the actuating element, wherein the actuating element is displaceable from a first position to a second position by the user's suction airstream, wherein the counting mechanism is coupled to the actuating element, wherein the counting mechanism is configured to change from the first state to the second state by displacing the actuating element from the first position to the second position, wherein the inhalation device further comprises a housing and an engaging member that is engageable to the housing, wherein the engaging member is a cap member, wherein the counting mechanism is configured to change from its second state to its first state by engaging the engaging member to the housing, and wherein the counting mechanism is configured such that a counted number is only incremented or decremented by engaging the engaging member to the housing if the counting mechanism had been altered to its second state beforehand.

14. Inhalation device according to claim 13, wherein the counting mechanism is enabled to increment or decrement the counted number only when the counting mechanism is in its second state.

15. Inhalation device according to claim 13, wherein the counting mechanism is configured such that a change from the second state to the first state increments or decrements the counted number.

16. Inhalation device according to claim 13, wherein the counting mechanism comprises a counting member enabled to interact with the interaction member for incrementing or decrementing the counted number.

17. Inhalation device according to claim 16, further comprising a coupling member, wherein the interaction member is configured to act on the coupling member and the coupling member is enabled to transmit a movement of the interaction member to the counting member.

18. Inhalation device according to claim 17, wherein the coupling member comprises a toothed wheel, and wherein the interaction member is configured to slide along a tooth of the toothed wheel when the counting mechanism is altered from the first state to the second state.

19. Inhalation device according to claim 17, wherein the coupling member and the counting member are formed by the same element.

20. Inhalation device according to claim 13, wherein the interaction member is deflectable.

21. Inhalation device according to claim 20, configured such that the interaction member is deflected in the first state of the counting mechanism and wherein the interaction member is undeflected in the second state of the counting mechanism.

22. Inhalation device according to claim 13, wherein the counting mechanism is in its first state when the engaging member is engaged to the housing.

23. Inhalation device according to claim 13, comprising a balancing member, wherein the interaction member and the balancing member are arranged at the actuating element such that the balancing member is arranged opposite to the interaction member.

24. Inhalation device according to claim 13, wherein the housing comprises a window showing the counted number of the counting mechanism.
